(19) United States
(12) Patent Application Publication
(10) Pub. No.: US 2026/0078234 A1
(43) Pub. Date: Mar. 19, 2026

(54) IMPROVED METHOD FOR PRODUCING ALPHA-BETA-UNSATURATED CARBOXYLIC ACIDS FROM POLY(3-HYDROXYALKANOATE)

(71) Applicant: Arkema France, Colombes (FR)

(72) Inventors: Yves CABON, Saint Avold Cedex (FR); Jean-Jacques GESLOT, Saint Avold Cedex (FR); Guillaume SABOT, Saint Avold Cedex (FR)

(21) Appl. No.: 19/108,486

(22) PCT Filed: Sep. 4, 2023
(86) PCT No.: PCT/EP2023/074176

§ 371 Date: Mar. 4, 2025

(30) Foreign Application Priority Data
Sep. 6, 2022 (FR) 2208914

(51) Int. Cl. C08J 11/12; C07C 51/43; C07C 51/44; C07C 51/48

(52) U.S. Cl. CPC C08J 11/12; C07C 51/43; C07C 51/44; C07C 51/48; C08J 2367/04

(57) ABSTRACT

The present invention relates to a method for producing α-β-unsaturated carboxylic acids by thermolysis of poly(3-hydroxyalkanoate), while limiting clogging associated with the unintentional condensation of hot α-β-unsaturated carboxylic acid vapours generated on walls in the method and with the subsequent formation of solids via a radical polymerisation reaction. The invention is based on the introduction of a radical polymerisation inhibitor into the thermolysis reactor and on the use of specific thermolysis conditions that make this inhibitor partially volatile during the thermolysis of the poly(3-hydroxyalkanoate).

### TECHNICAL FIELD

The present invention relates to a process for producing α,β-unsaturated carboxylic acids by thermolysis of poly(3-hydroxyalkanoate), while limiting the fouling phenomena associated with unintended condensation, on the walls of the process, of the hot α,β-unsaturated carboxylic acid vapors generated and with the subsequent formation of solids by a radical polymerization reaction. The invention is based on the introduction into the thermolysis reactor of a radical polymerization inhibitor and the use of specific thermolysis conditions making this inhibitor partially volatile during the thermolysis of the poly(3-hydroxyalkanoate). Thus, in the event of unintended condensation, on the walls of the process, of the hot α,β-unsaturated carboxylic acid vapors generated, the radical polymerization inhibitor also condenses and protects the liquid phase that is formed from a radical polymerization reaction.

Prior art and technical problem Nowadays, α,β-unsaturated carboxylic acids are produced industrially mainly from raw materials of fossil origin. For example, acrylic acid is obtained by oxidation of propylene, or methacrylic acid can be obtained by oxidation of isobutylene.

There is strong market demand for these α,β-unsaturated carboxylic acids, which are used as monomers in numerous applications, to be obtained from biobased raw materials. These biobased raw materials are derived from renewable organic matter (biomass) of biological origin (microorganisms, plants or animals).

One possible way of obtaining these α,β-unsaturated carboxylic acids is the thermolysis at temperatures of 150° C. to 300° C. of the corresponding poly(3-hydroxyalkanoates), according to the following reaction:

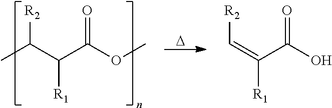

R₁═H or alkyl and R₂═H or alkyl; n is a number greater than 30
If R₁═R₂═H:
   poly(3-hydroxyalkanoate)=poly(3-hydroxypropionate) (P3HP);
   α,β-unsaturated carboxylic acid=propenoic acid (acrylic acid).
If R₁=methyl and R₂═H:
   poly(3-hydroxyalkanoate)=poly(3-hydroxyisobutyrate) (P3HiB);
   α,β-unsaturated carboxylic acid=isobutenoic acid (methacrylic acid).
If R₁═H and R₂=methyl:
   poly(3-hydroxyalkanoate)=poly(3-hydroxybutyrate) (P3HB);
   α,β-unsaturated carboxylic acid=but-2-enoic acid (crotonic acid).
If R₁═H and R₂=ethyl:
   poly(3-hydroxyalkanoate) is poly(3-hydroxyvalerate) (P3HV);
   α,β-unsaturated carboxylic acid=pent-2-enoic acid.

These poly(3-hydroxyalkanoates) can themselves be obtained beforehand by chemical transformations of raw materials of fossil origin, but also by fermentation of biomass.

One potential problem with any process for manufacturing α,β-unsaturated carboxylic acids is that these compounds can easily polymerize radically when they are hot and in the liquid phase. This is true in liquid phases that are formed deliberately, such as liquid phases present in a distillation column, in a reactor or in a condenser, but it can also occur in liquid phases that are formed unintentionally, such as those formed during unintended condensation of hot vapors on a wall with a cold spot. The usual consequence of this process is the deposition of solid polymers in the plant equipment, which eventually cause blockages and make it necessary to shut down the plant for cleaning, which is difficult and costly in terms of non-productive downtime.

To reduce these drawbacks, radical polymerization inhibitors are conventionally added at all stages of the production process, i.e. at the synthesis stage and at the purification stages.

For example, during the industrial manufacture of acrylic acid (AA), radical polymerization inhibitors are added to the absorption column for absorbing into water the AA vapors from the catalytic oxidation of propylene, and then to each distillation column and finally to the end product.

Radical polymerization inhibitors conventionally used in these manufacturing processes are phenol derivatives such as hydroquinone (HQ) and derivatives thereof such as hydroquinone methyl ether (HQME), 2,6-di-tert-butyl-4-methylphenol (BHT) or 2,4-dimethyl-6-tert-butylphenol (Topanol A); phenothiazine and derivatives thereof; nitroxide compounds such as 4-hydroxy-2,2,6,6-tetramethylpiperidin-1-oxyl (4-OH-TEMPO); and amino compounds such as para-phenylenediamine derivatives.

One drawback of these inhibitors is that they are usually considered non-volatile under the conditions for production of α,β-unsaturated carboxylic acid. In order to be present in all the liquid phases containing α,β-unsaturated acid, they must therefore be injected into the reaction, but also into the feeds, boilers, condensers and refluxes of the purification equipment. Sprays of inhibitors in solution can also be used to protect all surfaces on which hot α,β-unsaturated acid vapors are capable of unintentionally condensing. This problem is well known to those skilled in the art; for example, during the industrial purification of acrylic acid by distillation, polymerization inhibitors are added to the feed, the condenser and reflux of the distillation column but also frequently atomized in the form of sprays to protect the dome, gooseneck, manholes, or any other column component where acrylic acid vapors are capable of condensing.

Document EP 2398832 describes another solution aimed at preventing polymerization, including in the event of accidental unintended condensation of AA vapors. It uses a second type of inhibitor, referred to as a fugitive inhibitor, that is to say having a volatility, under the production operating conditions, which is similar to that of the α,β-unsaturated acid, here acrylic acid. This inhibitor is then present in the gas phase and condenses at the same time as the acrylic acid vapors during an unintended condensation. However, these polymerization inhibitors, which are nitrosobenzene derivatives, have the drawback of being toxic.

The problem of polymerization during the production of α,β-unsaturated carboxylic acids is also present when these acids are produced by thermolysis of the corresponding poly(3-hydroxyalkanoate).

U.S. Pat. No. 2,568,636 describes the thermolysis of poly(3-hydroxypropionate) (P3HP) to form acrylic acid (AA) at temperatures between 130° C. and 300° C. and the use of triarylphosphates to limit the polymerization of AA in the thermolysis reactor. U.S. Pat. No. 3,002,017 describes a similar thermolysis in which AA vapors are absorbed in cold AA in order to limit the polymerization during the condensation stage.

U.S. Pat. No. 9,115,070 describes the thermolysis of P3HP to form AA using a tertiary amine catalyst to reduce the reaction temperature. Conventional polymerization inhibitors, such as phenothiazine (PTZ), can potentially be used in the reaction medium in an amount of from 10 to 1000 ppm by weight with respect to P3HP in order to reduce the phenomena of polymerization of AA formed in the thermolysis medium.

U.S. Pat. No. 10,065,914 describes the thermolysis of P3HP to form AA at temperatures of 100° C. to 300° C., using a sodium acrylate catalyst to reduce the reaction temperature and thus limit the risks of polymerization of the AA formed in the thermolysis medium. The use of polymerization inhibitors, such as PTZ and HQME, in the liquid phase of the thermolysis reactor and their deliberate introduction into the liquid phases of a distillation or condenser also make it possible to reduce the polymerization phenomena.

However, the processes of the prior art have a major drawback. They describe how to reduce the risks associated with the polymerization of liquid phases of α,β-unsaturated acids by injecting a polymerization inhibitor into these deliberately formed liquid phases (thermolysis medium, condenser, liquid phase of a distillation column, etc.), but do not provide a solution in the case where the liquid phase is formed unintentionally, for example during the unwanted condensation of α,β-unsaturated acid on a “cold spot” of a plant. The use of sprays for spraying these inhibitors on all the walls of an industrial system is certainly possible but complex to implement. The use of non-conventional inhibitors, such as nitrosobenzene derivatives, is also complex to implement industrially.

The inventors have now surprisingly discovered that it is possible to drastically reduce the polymerization phenomena associated with the unintended condensation of hot vapors in a process for synthesizing α,β-unsaturated carboxylic acid from poly(3-hydroxyalkanoate) without using a non-conventional inhibitor. Specifically, the parameters of the thermolysis reaction of poly(3-hydroxyalkanoate) to give α,β-unsaturated carboxylic acid can be adjusted in order to obtain a significant volatility of some conventional polymerization inhibitors. These inhibitors are then present in the gas phase and condense at the same time as the α,β-unsaturated carboxylic acid during an unintended condensation on a cold spot, instantly protecting the liquid phase formed. There is no need for a complex system for delivering inhibitor at several points of the process, nor for expensive and toxic inhibitors.

Consequently, the invention proposes to provide a simple and easily implementable solution for reducing the fouling phenomena and to thus maintain high reliability and increased productivity in processes for producing α,β-unsaturated carboxylic acids from poly(3-hydroxypropionate).

### SUMMARY OF THE INVENTION

The invention relates to a process for producing α,β-unsaturated carboxylic acids by thermolysis of poly(3-hydroxyalkanoate) carried out in a thermolysis reactor from which the carboxylic acid vapors generated join a condenser, in the presence of one or more polymerization inhibitors, characterized in that the pressure in the reactor is adjusted in order to be less than twice the vapor pressure of at least one of the inhibitors at the temperature at which the thermolysis is carried out.

According to the invention, the thermolysis conditions used (pressure and temperature) allow one of the polymerization inhibitors to be significantly volatile and thus to obtain the desired effect.

According to various implementations, said process comprises the following features, where appropriate in combination. The contents indicated are expressed by weight, unless otherwise indicated. In the ranges of values indicated, the limits are included.

According to one embodiment, the poly(3-hydroxyalkanoate) used in the thermolysis process comprises a single type of 3-hydroxyalkanoate units and the product formed is therefore composed of a single α,β-unsaturated carboxylic acid.

According to one embodiment, the poly(3-hydroxyalkanoate) used in the thermolysis process comprises several different 3-hydroxyalkanoate units and the product formed is therefore composed of a mixture of various α,β-unsaturated carboxylic acids. Examples of P3HA copolymers are poly-3-hydroxybutyrate-co-3-hydroxypropionate (poly-3HB-co-3HP) or poly-3-hydroxybutyrate-co-3-hydroxyvalerate (poly-3HB-co-3HV).

In one embodiment, the poly(3-hydroxyalkanoate) used in the thermolysis process is obtained from raw materials of fossil origin.

According to one embodiment, the poly(3-hydroxyalkanoate) used in the thermolysis process is obtained from raw materials of renewable origin or at least partly of renewable origin. According to this embodiment, the poly(3-hydroxyalkanoate) is more than 50% by weight, preferably more than 80% by weight, advantageously 100% by weight of renewable origin.

According to one embodiment, the poly(3-hydroxyalkanoate) used in the thermolysis process is obtained by chemical reaction, for example P3HP is obtained by polymerization of β-propiolactone, which is itself obtained from ethylene oxide and carbon monoxide.

According to one embodiment, the poly(3-hydroxyalkanoate) used in the thermolysis process is obtained by biological reaction, in particular by fermentation.

According to one embodiment, the poly(3-hydroxyalkanoate) used in the thermolysis process is purified prior to the thermolysis reaction.

According to one embodiment, the poly(3-hydroxyalkanoate) used in the thermolysis process is used without prior purification, in particular without separation of the cell membrane, if it was obtained by fermentation.

In one embodiment, the poly(3-hydroxyalkanoate) is obtained inside a cell by a fermentation reaction, the biomass is washed and dried, but the poly(3-hydroxyalkanoate) is not separated from the cell membrane before the thermolysis step.

In one embodiment, the poly(3-hydroxyalkanoate) is obtained inside a cell by a fermentation reaction, the biomass is washed and dried and the poly(3-hydroxyalkanoate) is separated from the cell membrane before the thermolysis step, for example by extraction.

According to one embodiment, the poly(3-hydroxyalkanoate) thermolysis reaction takes place in the absence of solvent, the product then being in solid form or in the melt state.

According to one embodiment, the poly(3-hydroxyalkanoate) thermolysis reaction takes place in solution.

According to one embodiment, the poly(3-hydroxyalkanoate) thermolysis reaction takes place in suspension.

According to one embodiment, the poly(3-hydroxyalkanoate) thermolysis reaction takes place in batch mode.

According to one embodiment, the poly(3-hydroxyalkanoate) thermolysis reaction takes place continuously.

According to one embodiment, the poly(3-hydroxyalkanoate) thermolysis reaction takes place in the absence of catalyst.

The polymerization inhibitors used in the process according to the invention are chosen from inhibitors conventionally used in existing industrial processes for producing α,β-unsaturated carboxylic acids. These include phenol derivatives such as hydroquinone (HQ) and derivatives thereof; phenothiazine and derivatives thereof; nitroxide compounds; and amino compounds such as para-phenylenediamine derivatives.

According to one embodiment, the poly(3-hydroxyalkanoate) contains the 3-hydroxypropionate unit and at least one of the α,β-unsaturated carboxylic acids produced is acrylic acid.

According to one embodiment, the poly(3-hydroxyalkanoate) is poly(3-hydroxypropionate) and the α,β-unsaturated carboxylic acid produced is acrylic acid.

According to one embodiment, the poly(3-hydroxyalkanoate) contains the 3-hydroxybutyrate unit and at least one of the α,β-unsaturated carboxylic acids produced is crotonic acid.

According to one embodiment, the poly(3-hydroxyalkanoate) is poly(3-hydroxybutyrate) and the α,β-unsaturated carboxylic acid produced is crotonic acid.

According to one embodiment, the poly(3-hydroxyalkanoate) contains the 3-hydroxyisobutyrate unit and at least one of the α,β-unsaturated carboxylic acids produced is methacrylic acid.

In one embodiment, the poly(3-hydroxyalkanoate) is poly(3-hydroxyisobutyrate) and the α,β-unsaturated carboxylic acid produced is methacrylic acid.

Another subject of the invention relates to a process for purifying the α,β-unsaturated carboxylic acid(s) obtained by the poly(3-hydroxyalkanoate) thermolysis process carried out at a pressure less than twice the vapor pressure of at least one polymerization inhibitor at the thermolysis temperature, characterized in that it comprises a step of condensing the vapors of the α,β-unsaturated carboxylic acid(s) thus obtained, followed by one or more purification steps.

The present invention meets the need expressed in the prior art. It makes it possible to prevent the risks of fouling due to the unintended condensation of α,β-unsaturated carboxylic acid vapors on cold spots in the case of the generation of α,β-unsaturated carboxylic acids by thermolysis of poly(3-hydroxypropionate). In particular, the invention makes it possible to protect the area located between the thermolysis reactor and the condenser. Owing to the polymerization inhibitor being rendered volatile in the thermolysis medium, it will be present in the gas phase throughout the part of the plant where the α,β-unsaturated carboxylic acid(s) are in the gas phase. The invention also makes it possible to avoid the formation of polymers in the reaction medium.

The invention will now be described in more detail in the description that follows.

### DETAILED DESCRIPTION OF THE INVENTION

The invention aims to produce α,β-unsaturated carboxylic acids on an industrial scale by thermolysis of poly(3-hydroxyalkanoate), without being faced with the problem of fouling of the equipment used, due to the polymerization of the α,β-unsaturated carboxylic acid vapors when condensing on cold spots of the equipment.

The invention proposes to provide a process that makes it possible to reduce or eliminate this risk of fouling. The invention is based on the addition of a polymerization inhibitor and the choice of pressure and temperature conditions in the poly(3-hydroxyalkanoate) thermolysis reactor, so that the inhibitor has significant volatility under the reaction conditions. Typically, the pressure in the reactor is adjusted to be less than twice the vapor pressure of the inhibitor at the thermolysis temperature.

The term “thermolysis” of the poly(3-hydroxyalkanoate) means its chemical decomposition to α,β-unsaturated carboxylic acid obtained under the effect of temperature. This term is synonymous with pyrolysis.

According to the IUPAC, “saturation vapor pressure” is the pressure exerted by a pure substance (at a given temperature) in a system containing only the vapor and condensed phase (liquid or solid) of the substance. (Pure and Applied Chemistry, 1990, Volume 62, No. 11, pp. 2167-2219 & Glossary of atmospheric chemistry terms (Recommendations 1990), page 2212).

In the description of the invention, the term “vapor pressure” will be used in the same sense as the term “saturation vapor pressure”. Also, the term “vapor pressure” is synonymous with “vapor tension”.

In the poly(3-hydroxyalkanoate) thermolysis process according to the invention, the poly(3-hydroxyalkanoate) is heated to a temperature of from 130° C. to 300° C., preferably from 170° C. to 230° C.

The reaction medium in the thermolysis reactor comprises at least one polymerization inhibitor, in a proportion notably of from 50 ppm to 5% by weight, in particular from 0.01% to 3% by weight, relative to the weight of the poly(3-hydroxyalkanoate). When there are two or more inhibitors, their overall content does not exceed 5% by weight.

The polymerization inhibitors are selected from inhibitors conventionally used in existing industrial processes for the production of α,β-unsaturated carboxylic acids. These include phenol derivatives such as hydroquinone (HQ) and derivatives thereof such as hydroquinone methyl ether (HQME), 2,6-di-tert-butyl-4-methylphenol (BHT) or 2,4-dimethyl-6-tert-butylphenol (Topanol A); phenothiazine and derivatives thereof; nitroxide compounds such as 4-hydroxy-2,2,6,6-tetramethylpiperidin-1-oxyl (4-OH-TEMPO); and amino compounds such as para-phenylenediamine derivatives.

In the poly(3-hydroxyalkanoate) thermolysis process according to the invention, the temperature and pressure conditions in the thermolysis reactor are chosen so that the α,β-unsaturated carboxylic acid(s) formed are in vapor form and at least one of the inhibitors is volatile. This is obtained when the pressure in the reactor is less than twice the vapor pressure of one of the inhibitors at the thermolysis temperature.

According to one embodiment, at least one of said polymerization inhibitors is hydroquinone methyl ether (HQME).

By way of example, HQME has a vapor pressure of:
20 kPa at 190° C.; the pressure in the reactor is adjusted to below 40 kPa for thermolysis at 190° C.;
28.5 kPa at 200° C.; the pressure in the reactor is adjusted to below 57 kPa for thermolysis at 200° C.;
39 kPa at 210° C.; the pressure in the reactor is adjusted to below 78 kPa for thermolysis at 210° C.

The process according to the invention thus makes it possible to adopt specific pressure conditions in order to obtain volatility of the inhibitor and thus protect the operation in the event of undesired condensation of hot α,β-unsaturated carboxylic acid vapors on a cold spot of one of the walls of the equipment.

According to one embodiment of the invention, the thermolysis reaction is carried out in the presence of a solvent, either in solution or in suspension. In order to limit the vaporization of the solvent with the α,β-unsaturated carboxylic acid vapors generated during the thermolysis of the poly(3-hydroxyalkanoate), the solvent is chosen so that its vapor pressure at the thermolysis temperature of the poly(3-hydroxyalkanoate) is less than three quarters of the pressure at which the thermolysis is carried out.

According to one embodiment, for operating conditions of 200° C. and 20 kPa, the solvent must have a vapor pressure at 200° C. of less than 15 kPa and can therefore be chosen from:
higher alkanes containing more than 14 carbon atoms; by way of example, the vapor pressure of n-hexadecane (C16) at 200° C. is 10 kPa. When the solvent is an alkane, the thermolysis reaction takes place in suspension.
fatty acids containing more than 8 carbon atoms; by way of example, the vapor pressure of capric acid (C10) at 200° C. is 11.2 kPa. When the solvent is a fatty acid, the thermolysis reaction takes place in suspension.
polyglycol dimethyl ethers (glymes) starting from tetraglyme; by way of example, the vapor pressure of tetraglyme at 200° C. is 10.2 kPa. When the solvent is a glyme, the thermolysis reaction takes place in suspension.
sulfolane, which has a vapor pressure at 200° C. of 10.3 kPa. When the solvent is sulfolane, the thermolysis reaction takes place in solution.

According to one embodiment, when the poly(3-hydroxyalkanoate) thermolysis reaction is carried out in suspension or solution in a solvent, the operating pressure is between 1.5 times the vapor pressure of the solvent at the thermolysis temperature and twice the vapor pressure of at least one inhibitor at the thermolysis temperature.

The preferred operating pressure for the poly(3-hydroxyalkanoate) thermolysis reaction is just below the vapor pressure of the inhibitor.

According to a preferred embodiment, the polymerization inhibitor is hydroquinone methyl ether and the operation is carried out in solution in a solvent such as sulfolane or tetraglyme.

According to one embodiment, the thermolysis of the poly(3-hydroxyalkanoate) takes place in the absence of a catalyst. The use of catalysts makes it possible to accelerate the thermolysis kinetics and/or to reduce the thermolysis temperature. However, the use of a catalyst makes the process more complex and more difficult to implement on an industrial scale.

The invention also relates to a process for purifying the α,β-unsaturated carboxylic acid(s) obtained by the poly(3-hydroxyalkanoate) thermolysis process carried out at a pressure less than twice the vapor pressure of at least one polymerization inhibitor at the thermolysis temperature, characterized in that it comprises a step of condensing the vapors of the α,β-unsaturated carboxylic acid(s) thus obtained, followed by one or more purification steps. The purification operations may generally include distillations, liquid/liquid extractions, separations using a film evaporator, or crystallizations, or a combination of these techniques.

The examples below illustrate the present invention without, however, limiting the scope thereof.

### EXPERIMENTAL SECTION

Tests of the thermolysis of poly(3-hydroxypropionate) (P3HP) in order to generate acrylic acid (AA) are performed in a laboratory assembly. 2 g of pure P3HP are introduced into a 25 ml two-neck round-bottom flask.

An inhibitor (PTZ or HQME) is optionally added in an amount of 20 mg.

A solvent is optionally added in an amount of 10 g.

The side neck of the round-bottom flask is equipped with a thermometer to monitor the reaction temperature. The upper neck of the round-bottom flask is equipped with a separation bridge leading to a water-cooled side condenser, itself leading to a receiver consisting of a second 25 ml round-bottom flask. A tap between the condenser and the receiver makes it possible to establish a reduced pressure in the assembly.

At the beginning of the experiment, the system is placed under the desired pressure and then the flask containing the P3HP, and optionally the inhibitor and/or the solvent, is placed in a heating system that enables the desired thermolysis temperature to be established (oil bath or electric heating mantle). The receiver is cooled by an ice bath. The separation bridge between the thermolysis round-bottom flask and the side condenser is left uninsulated to simulate the existence of cold spots.

As soon as the thermolysis reactor reaches more than 170° C., the formation of AA vapors is observed, which vapors condense mainly in the side condenser but also on the cold spots of the separation bridge. After 4 h of heating, the formation of AA vapors in the thermolysis reactor tapers off and the experiment is then stopped.

The degree of fouling of the separation bridge, representing the zone of unintentional condensation of AA on cold spots in an industrial plant, is then judged visually. The AA recovered in the receiver is also analyzed by gas chromatography in order to verify the presence or absence of the inhibitor optionally introduced into the thermolysis reactor, testifying to its volatility or non-volatility under the experimental conditions tested.

The main results obtained are presented in table 1.

Comparative tests 1, 2, 8, 9, 15 and 16, carried out without any polymerization inhibitor, show heavy fouling of the separation bridge where hot AA vapors condense on cold spots, whether they are carried out without solvent (1, 2), in suspension (8, 9) or in solution (15, 16) and at atmospheric pressure (1, 8, 15) or under a reduced pressure of 20 kPa (2, 9, 16).

Comparative tests 3, 6, 7, 10, 13, 14, 17, 20 and 21, carried out in the presence of a polymerization inhibitor but under an operating pressure in the assembly of more than twice the vapor pressure of the inhibitor at the thermolysis temperature, also show heavy fouling of the separation bridge where hot AA vapors condense on cold spots, whether they are carried out without solvent (3, 6, 7), in suspension (10, 13, 14) or in solution (17, 20, 21). It is also noted that no traces of the inhibitor are found in the AA recovered in the receiver, a sign that it was not volatile under the thermolysis operating conditions.

Tests 4, 5, 11, 12, 18 and 19 according to the invention, carried out in the presence of a polymerization inhibitor under an operating pressure in the assembly of less than twice the vapor pressure of the inhibitor, show a notable reduction in the fouling of the separation bridge where hot AA vapors condense on cold spots, whether they are carried out in bulk (4, 5), in suspension (11, 12) or in solution (18, 19). Also noted is the presence of inhibitor in the AA recovered in the receiver, at least in trace amounts, a sign that it was volatile under the thermolysis operating conditions. The reduction in fouling, and also the presence of the inhibitor in the AA formed, is small but significant when the operating pressure is just below twice the vapor pressure of the inhibitor at the thermolysis temperature (4, 11, 18), and more marked when the operating pressure is below the vapor pressure of the inhibitor at the thermolysis temperature (5, 12, 19).

TABLE 1

| | | Inhibitor | | Solvent | | Operating | | |
|---|---|---|---|---|---|---|---|---|
| | | vapor | | vapor | | pressure <2× | Fouling | |
| | | pressure | | pressure | Operating | inhibitor | of | |
| | | at 200° C. | | at 200° C. | pressure | vapor | critical | Inhibitor |
| Test | Inhibitor | (kPa) | Solvent | (kPa) | (kPa) | pressure | area | in AA |
| 1 | No | — | No | — | 101 | No | Heavy | — |
| 2 | No | — | No | — | 20 | No | Heavy | — |
| 3 | HQME | 28.5 | No | — | 101 | No | Heavy | No |
| 4 | HQME | 28.5 | No | — | 55 | No | Light | Trace |
| | | | | | | | | amounts |
| 5 | HQME | 28.5 | No | — | 20 | Yes | None | Yes |
| 6 | PTZ | 0.7 | No | — | 101 | No | Heavy | No |
| 7 | PTZ | 0.7 | No | — | 20 | No | Heavy | No |
| 8 | No | — | C18 | 3.7 | 101 | No | Heavy | — |
| 9 | No | — | C18 | 3.7 | 20 | No | Heavy | — |
| 10 | HQME | 28.5 | C18 | 3.7 | 101 | No | Heavy | No |
| 11 | HQME | 28.5 | C18 | 3.7 | 55 | No | Light | Trace |
| | | | | | | | | amounts |
| 12 | HQME | 28.5 | C18 | 3.7 | 20 | Yes | None | Yes |
| 13 | PTZ | 0.7 | C18 | 3.7 | 101 | No | Heavy | No |
| 14 | PTZ | 0.7 | C18 | 3.7 | 20 | No | Heavy | No |
| 15 | No | — | Sulfolane | 10.3 | 101 | No | Heavy | — |
| 16 | No | — | Sulfolane | 10.3 | 20 | No | Heavy | — |
| 17 | HQME | 28.5 | Sulfolane | 10.3 | 101 | No | Heavy | No |
| 18 | HQME | 28.5 | Sulfolane | 10.3 | 55 | No | Light | Trace |
| | | | | | | | | amounts |
| 19 | HQME | 28.5 | Sulfolane | 10.3 | 20 | Yes | None | Yes |
| 20 | PTZ | 0.7 | Sulfolane | 10.3 | 101 | No | Heavy | No |
| 21 | PTZ | 0.7 | Sulfolane | 10.3 | 20 | No | Heavy | No |

Additional tests 22 to 31 (table 2), carried out in solvent at 200° C. and 20 kPa in the presence of HQME, i.e. under the conditions of the invention, show an absence of fouling of the separation bridge where hot AA vapors condense on cold spots. Also noted is the presence of inhibitor in the AA recovered in the receiver, a sign that it is volatile under the thermolysis operating conditions.

They make it possible to demonstrate the importance to be given to the choice of solvent during a poly(3-hydroxyalkanoate) thermolysis carried out according to the invention in a solvent medium. Thus, if the vapor pressure of the solvent at the thermolysis temperature (here 200° C.) is not less than three quarters of the operating pressure (here 20 kPa, i.e. a vapor pressure of the solvent at 200° C. of less than 15 kPa), the AA recovered in the receiver is heavily polluted by the solvent used (22, 26, 29). This phenomenon is drastically limited when using a solvent with a vapor pressure at the thermolysis temperature of less than three quarters of the operating pressure (23, 24, 25, 27, 28, 30, 31).

C14=n-tetradecane; C16=n-hexadecane; C18=n-octadecane; C20=n-eicosane
C8 acid=octanoic acid; C10 acid=decanoic acid; C12 acid=dodecanoic acid

TABLE 2

| | | Inhibitor | | Solvent | | Operating | | | | |
|---|---|---|---|---|---|---|---|---|---|---|
| | | vapor | | vapor | | pressure <2× | Solvent vapor | Fouling | | |
| | | pressure | | pressure | Operating | inhibitor | pressure <0.75× | of | | |
| | | at 200° C. | | at 200° C. | pressure | vapor | operating | critical | Inhibitor | Solvent |
| Test | Inhibitor | (kPa) | Solvent | (kPa) | (kPa) | pressure | pressure | area | in AA | in AA |
| 22 | HQME | 28.5 | C14 | 24.5 | 20 | Yes | No | None | Yes | >20% |
| 23 | HQME | 28.5 | C16 | 9.4 | 20 | Yes | Yes | None | Yes | <0.2% |
| 24 | HQME | 28.5 | C18 | 3.7 | 20 | Yes | Yes | None | Yes | <0.2% |
| 25 | HQME | 28.5 | C20 | 1.5 | 20 | Yes | Yes | None | Yes | <0.2% |
| 26 | HQME | 28.5 | C8 acid | 30 | 20 | Yes | No | None | Yes | >20% |
| 27 | HQME | 28.5 | C10 acid | 11.2 | 20 | Yes | Yes | None | Yes | <0.2% |
| 28 | HQME | 28.5 | C12 acid | 4.3 | 20 | Yes | Yes | None | Yes | <0.2% |
| 29 | HQME | 28.5 | Triglyme | 62.2 | 20 | Yes | No | None | Yes | >20% |
| 30 | HQME | 28.5 | Tetraglyme | 10.2 | 20 | Yes | Yes | None | Yes | <0.2% |
| 31 | HQME | 28.5 | Sulfolane | 10.3 | 20 | Yes | Yes | None | Yes | <0.2% |

1. A process for producing α,β-unsaturated carboxylic acids, the process comprising:
thermolysis of poly(3-hydroxyalkanoate) carried out in a reactor in the presence of at least one polymerization inhibitor, characterized in that an operating pressure in the reactor, at a thermolysis temperature, is less than twice the vapor pressure of said at least one polymerization inhibitor.

2. The process as claimed in claim 1, wherein the thermolysis temperature is between 130° C. and 300° C.

3. The process as claimed in claim 1, wherein the at least one polymerization inhibitor is present in an amount from 0.005% to 5% by weight relative to a weight of the poly(3-hydroxyalkanoate).

4. The process as claimed in claim 1, wherein said at least one polymerization inhibitor is a compound selected from phenolic derivatives, phenothiazine derivatives, nitroxide derivatives or para-phenylenediamine derivatives.

5. The process as claimed in claim 1, wherein said at least one polymerization inhibitor is hydroquinone methyl ether (MEHQ).

6. The process as claimed in claim 1, wherein the poly(3-hydroxyalkanoate) is of petrochemical origin or at least partly of renewable origin.

7. The process as claimed in claim 1, wherein the poly(3-hydroxyalkanoate) is more than 50% by weight of renewable origin.

8. The process as claimed in claim 1, wherein the poly(3-hydroxyalkanoate) is obtained by a chemical reaction.

9. The process as claimed in claim 7, wherein the poly(3-hydroxyalkanoate) is obtained by fermentation.

10. The process as claimed in claim 9, wherein the poly(3-hydroxyalkanoate) is separated from a biological medium prior to the thermolysis step.

11. The process as claimed in claim 1, wherein the thermolysis of poly(3-hydroxyalkanoate) is carried out in the absence of solvent.

12. The process as claimed in claim 1, wherein the thermolysis of poly(3-hydroxyalkanoate) is carried out in suspension in a solvent or in solution in a solvent.

13. (canceled)

14. The process as claimed in claim 12, wherein the solvent has a vapor pressure, at the thermolysis temperature, of less than three quarters of the operating pressure.

15. The process as claimed in claim 13, wherein the solvent is sulfolane.

16. (canceled)

17. The process as claimed in claim 1, wherein the thermolysis of poly(3-hydroxyalkanoate) is carried out in batch mode or continuously.

18. (canceled)

19. The process as claimed in claim 1, wherein the poly(3-hydroxyalkanoate) contains a 3-hydroxypropionate unit and at least one of the α,β-unsaturated carboxylic acids produced is acrylic acid.

20. (canceled)

21. The process as claimed in claim 1, wherein the poly(3-hydroxyalkanoate) contains a 3-hydroxybutyrate unit and at least one of the α,β-unsaturated carboxylic acids produced is crotonic acid.

22. (canceled)

23. The process as claimed in claim 1, wherein the poly(3-hydroxyalkanoate) contains a 3-hydroxyisobutyrate unit and at least one of the α,β-unsaturated carboxylic acids produced is methacrylic acid.

24. (canceled)

25. A process for producing α,β-unsaturated carboxylic acids as claimed in claim 1, further comprising a step of condensing the produced α,β-unsaturated carboxylic acids, followed by one or more purification steps selected from distillation, liquid/liquid extraction, separation using a film evaporator, or crystallization, or a combination of these techniques.
